(19) United States
(12) Patent Application Publication
(10) Pub. No.: US 2018/0235982 A1
(43) Pub. Date: Aug. 23, 2018

(54) Treatment for Spirochete borne illness

(71) Applicant: Michael Earl Drabinsky, Walnut Creek, CA (US)

(72) Inventor: Michael Earl Drabinsky, Walnut Creek, CA (US)

(21) Appl. No.: 15/436,853

(22) Filed: Feb. 19, 2017

(51) Int. Cl. A61K 31/65; A61K 31/4164; A61K 47/28; A61K 31/538; A61K 31/42; A61K 31/43; A61K 31/505; A61K 9/00

(52) U.S. Cl. CPC A61K 31/65; A61K 31/4164; A61K 47/28; A61K 31/538; A61K 9/0053; A61K 31/43; A61K 31/505; A61K 9/0014; A61K 31/42

(57) ABSTRACT

A method and pharmaceutical composition for treating Spirochete phylum borne illnesses administered to a patient in need of such treatment at least one of tinidazole concentration between 10 to 10,000 mg dissolving bacterial biofilm or other nitroimidazole class drugs in combination with minocycline concentration of approximately 5 to 600 mg killing a bacterial infection co-administered with one of the five Vitamin Ds.

### CROSS REFERENCE TO RELATED APPLICATIONS

This application claims the benefit of and incorporates by reference continuance in part U.S. provisional patent application Ser. No. 62/296,681 titled TINIDAZOLE DRUG COMBINATIONS FOR FIGHTING PATHOGENS filed Feb. 21, 2016.

### BACKGROUND OF THE INVENTION

### Field of the Invention

This invention relates to a combination of two or more short and long acting anti-pathogenic compounds, including tinidazole, which dissolve the protective biofilm barriers of microbes found within the blood vessel walls or any nearby tissue to treat Borellia, Babesia, and other biofilm producing pathogens and amoebas.

Borrelia is called the great imitator because it can imitate (cause) any disease anywhere in the body. For example, many diseases such as Diabetes, MS, ALS, Lupus, Rheumatoid Arthritis, Ehler's-Danlos type 3, Fibromyalgia, Alzheimer's, Dementia, Parkinson's, Autism, etc. are probably caused by Borrelia.

Prior to this invention, there was no cure for Lyme disease. There were only temporary treatments and palliative care. This is the only cure for Borrelia. There was also no way to culture or study Borrelia, thus explaining the significant lack of scientific data surrounding Borrelia diagnostics and treatment.

There is currently a debate within the medical community about early vs. late stage Lyme disease. My experiments have found that there is no substantial difference between early and late stage Lyme. Some people with ‘early’ Lyme feel better after taking up to a month's worth of doxycycline because the doxycycline kills the motile spirochete forms in the bloodstream. Usually people who are treated inadequately for early Lyme relapse later in life because no amount of tetracycline class antibiotics can destroy the biofilm. Lyme hides in biofilm and can come back after antibiotic therapy has been stopped.

The CDC is currently unable to detect Borrelia in late stage (chronic) Lyme and therefore attributes the symptoms to psychiatric diseases or rheumatoid arthritis. However, late stage Lyme can be cured with the antibiotic regimen described within.

Even though there are some ‘treatments’ that the CDC and other organizations recommend to treat various stages of Lyme disease. These recommendations are usually for up to one month of doxycycline or amoxicillin. While people may temporarily feel better when taking one of these antibiotics, it is usually because the active, motile spirochetes in the blood get killed. But they come back from cyst and biofilm forms at a later date, sometimes years later, and manifest as varying symptoms.

Microorganisms generally live attached to surfaces in many natural, industrial, and medical environments, encapsulated by extracellular substances including biopolymers and macromolecules. The resulting layer of slime-encapsulated microorganism is termed a biofilm. A biofilm is any group of microorganisms which cells stick to each other on a surface. These adherent cells are frequently embedded within a self-produced matrix of extracellular polymeric substance (EPS). Biofilms are the predominant mode of growth of bacteria in the natural environment, and bacteria growing in biofilms exhibit distinct physiological properties. Compared to their planktonically grown counterparts, the bacteria in a biofilm are more resistant to antibiotics, UV irradiation, detergents and the host immune response (Gristina et al. 1988. Journal of the American Medical Association, 259: 870-874; Stewart. 1994. Antimicrobial Agents & Chemotherapy, 38(5): 1052-1058; Costerton et al. 1995. Annu. Rev. Microbiol., 49: 71 1-745; Maira Litran et al. 2000. Journal of Applied Microbiology, 88: 243-247). A biofilm may include one or more microorganisms, including gram-positive and gram-negative bacteria, algae, protozoa, and/or yeast or filamentous fungi and viruses and/or bacteriophage. Examples of problematic biofilms are dental plaque, infections on medical implants, but also the initial fouling on ship hulls (Satuito et al. 1997. Hydrobiologia, 358: 275-280). Biofilms are attributed to the pathogenesis of many infections in humans. New strategies are required to disperse existing biofilm.

It is known in the art that biofilms can have, as a component, DNA (termed extracellular DNA or eDNA) although its function there remains unknown. Certain groups have sought to employ nuclease enzymes to disrupt biofilms.

There are two (2) basic methods for disrupting biofilm in a patient: 1) Dissolution via chemical means (such as tinidazole); and 2) Dissolution via physical means (such as a CVAC machine or rapid atmospheric decompression followed by recompression.

However, the process for atmospheric biofilm disruption only works one way. That means one has to go from high pressure to low pressure to high pressure. It does not work going from low pressure to high pressure, like in hyperbaric oxygen therapy. In other words, hyperbaric oxygen therapy (or SCUBA diving) will not disrupt biofilms.

Many types of microbes grow naturally in a biofilm context, such as bacteria, fungi, algae etc.

Borrelia, like the other microbes produces biofilm and has active spirochete forms. Also, Borrelia has cysts which are spirochetes balled up and encapsulated in biofilm. The spirochete form of Borrelia is very mobile and can penetrate deep into any body tissues including cartilage, tendons, and bones.

Tinidazole is the best molecule currently known for dissolving biofilms. Metronidazole (Flagyl) is in the same drug class as tinidazole and can work in a similar fashion, however Flagyl is not as effective as Tindamax for dissolving biofilms and Flagyl has more adverse side effects.

Other compounds such as nattokinase and serrapeptase can dissolve biofilms, however they are not very effective in a patient treatment setting even though nattokinase and serrapeptase are less expensive than Tindamax.

Tindamax eliminates biofilm from the body because atmospheric biofilm disruption does not reduce the total volume of biofilm in one's body, but rather, it breaks apart larger chunks of biofilm into smaller chunks.

Anti-microbials are used concurrently with biofilm disruption techniques to kill the microbes that are released when biofilm is broken apart or destroyed because when biofilm is dissolved, pathogenic organisms are usually released into the body or bloodstream. Otherwise, if biofilm is broken up too rapidly, several things can happen. First, pathogens may be released into the bloodstream so quickly that sepsis ensues which can be harmful (life-threatening) to the patient. Second, rapid physical ‘chopping’ of large biofilm chunks can break them apart into smaller chunks which can then block blood vessels causing a whole bunch of problems such as heart attack, stroke, TIA, seizure, organ damage/failure due to lack of blood flow, etc.

In order to prevent further harm to a patient, methods of getting rid of biofilm should gradually be ramped up so there is a slow, safe, controlled release of biofilm chunks. Also, there is usually a high density of individual pathogens within a single biofilm chunk that get released when conducting a treatment to get rid of biofilm.

Tinidazole is only good at destroying biofilm and it is very limited in killing actual organisms.

Although it may be considered obvious on the broader level to combine tinidazole with antibiotics for treating borellia or similar infections, in particular, the removal of biofilms currently poses significant problems since the bacteria present in the biofilm are highly resistant to many antimicrobial compounds. Furthermore, prior art methods for biofilm disruption involve compositions active against mainly only gram negative proteobacteria, and show very specific activity against a limited number of strains. This significantly limits their utility. Thus, there remains a need for new biofilm disruption and treatment involving compositions with improved properties.

### DESCRIPTION OF TIE PREFERRED EMBODIMENTS

This invention uses a minimum of two or three antibiotics for the treatment of Borrellia. One antibiotic is required to dissolve Borrelia biofilm. A second or third antibiotic is required to kill the active spirochetes.

The current invention uses the brand name Tindamax (tinidazole), marketed by Mission Pharmacal, or Fasigyn and Simplotan marketed by Pfizer, which attacks the biofilm that the bacteria use to protect themselves, rendering most drug treatments useless.

The chemical name for Tinidazole's is C₈H₁₃N₃O₄S.

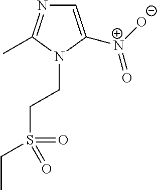 [SMILES: CCS(=O)(=O)CCn1c([N+](=O)[O-])cnc1C]

In prior art systems, the biofilm stops the drugs from reaching the bacteria, and this prevents their treatment regardless of which drugs are used. Tinidazole should be used to dissolve biofilms. Other nitroimidazole class drugs may be used instead of tinidazole to dissolve biofilm.

Minocycline is the best Borrelia antibiotic because it is lipophilic and penetrates most, if not all, body tissues including the central nervous system. Doxycycline may also be used, but doxycycline does not work as well because it does not penetrate all body tissues.

The chemical name for Minocycline is C₂₃H₂₇N₃O₇.

Vitamin D3 (cholecalciferol) is Borrelia's most usable form of food. Vitamin D3 is also lipophilic with a half-life of one to two weeks. Vitamin D3 may be co-administered with minocycline to enhance the uptake of minocycline, which enhances the efficacy of minocycline.

Minocycline's half-life is about 12-14 hours. A fluoroquinolone such as Levaquin is also recommended for co-administration. Levaquin is both water and lipid soluble and penetrates many body tissues pretty well. Other fluoroquinolones such as ciprofloxacin may be used, but do not work as well as Levaquin.

The chemical name for Levaquin is (−)-(S)-9-fluoro-2,3-dihydro-3-methyl-10-(4-methyl-1-piperazinyl)-7-oxo-7H-pyrido[1,2,3-de]-1,4-benzoxazine-6-carboxylic acid hemihydrate.

In children younger than eight, a penicillin class drug such as amoxicillin or bicillin may be used to substitute a tetracycline class drug due to bone (teeth) staining. However, in severe cases, or in children with a penicillin allergy, the benefit of minocycline may outweigh the downside of bone staining.

Sulfa class drugs such as Septra DS may be used, but usually with limited success and a high resistance rate. This medication is a combination of two antibiotics: sulfamethoxazole and trimethoprim.

Palliative support such as pain meds, glutathione, and rest may be necessary to accommodate herxing because with other Lyme co-infections, herxing can be severe.

Whilst the prior art recognized that antibiotics are used to treat borellia, babesia, and other biofilm producing pathogens and amoebas. However, none of the prior art used a Tinidazole drug combination for Treatment of Borellia et al to break down biofilms that pathogens protect themselves with, alongside antibiotics that can destroy the pathogens.

Thus, the invention provides a pharmaceutical composition for disrupting a biofilm or preventing biofilm formation comprising a Tinidazole drug combination.

In any of the pharmaceutical compositions described herein the composition can be formulated for oral administration.

In any of the pharmaceutical compositions described herein the composition can be formulated as a liquid, lotion, cream, spray, gel, ointment, or powder, and the like.

Any of the pharmaceutical compositions describe herein can be formulated for use in the treatment of a wide range of medical indications.

Where the composition is a pharmaceutical composition, said composition can be for administration to an animal patient. The animal patient can be a mammalian patient. The mammalian patient can be a human.

The invention also provides a method of disrupting a biofilm on a patient comprising contacting a biofilm on a patient with any of the pharmaceutical compositions described herein.

The patient can be an animal patient. The patient can be a mammalian patient. The patient can be a human.

In any of these methods the biofilm can comprise gram-positive bacteria.

Examples of formulations include topical lotions, creams, soaps, wipes, and the like. They may be formulated into liposomes, to reduce toxicity or increase bioavailability. Other methods for delivery include oral methods that entail encapsulation of the polypeptide or peptide in microspheres or proteinoids, aerosol delivery (e.g., to the lungs), or transdermal delivery (e.g., by iontophoresis or transdermal electroporation). Other routine methods of administration will be known to those skilled in the art.

Pharmaceutical formulations, containing any of the compositions described herein, suitable for oral administration may be provided in convenient unit forms including capsules, tablets, gels, pastes, ointments etc.

Preparations for parenteral administration of pharmaceutical formulations comprising compositions described herein include sterile aqueous or non-aqueous solutions, suspensions, and emulsions. Examples of non-aqueous solvents are propylene glycol, polyethylene glycol, vegetable oils (e.g., olive oil), and injectable organic esters such as ethyl oleate. Examples of aqueous carriers include water, saline, and buffered media, alcoholic/aqueous solutions, and emulsions or suspensions. Examples of parenteral vehicles include sodium chloride solution, Ringer's dextrose, dextrose and sodium chloride, lactated Ringer's, and fixed oils. Intravenous vehicles include fluid and nutrient replenishers, electrolyte replenishers (such as those based on Ringer's dextrose), and the like. Preservatives and other additives such as, other antimicrobial, antioxidants, chelating agents, inert gases and the like also can be included.

For topical administration to the epidermis, any of the pharmaceutical compositions may be formulated as an ointment, cream, or lotion. Ointments and creams, may, for example, be formulated with an aqueous or oily base with the addition of suitable thickening and/or gelling agents. Lotions may be formulated with an aqueous or oily base and will in general also contain one or more emulsifying agents, stabilizing agents, suspending agents, thickening agents, or coloring agents.

While there have been described what are presently believed to be the preferred embodiments of the invention, those skilled in the art will realize that various changes and modifications of another molecule or drug of a similar family may be made to the invention without departing from the spirit of the invention, and it is intended to claim all such changes and modifications as fall within the scope of the invention.

1. A method for treating Spirochete phylum borne illnesses comprising administering to a patient in need of such treatment at least one of tinidazole concentration between 10 to 10,000 mg dissolving bacterial biofilm in combination with minocycline concentration of approximately 5 to 600 mg killing a bacterial infection.

2. The method of claim 1 further comprising administering other nitroimidazole class drugs.

3. The method of claim 1 further comprising administering doxycycline.

4. The method of claim 1 further comprising administering one of the five Vitamin Ds coadministering with minocycline enhancing update and efficacy of the minocycline.

5. The method of claim 1 further comprising administering fluroquinolone as coadministrator.

6. The method of claim 1 further comprising pencillin class drugs for patient younger than eight years old preventing teeth staining.

7. The method of claim 1 further comprising administering sulfa class drugs.

8. A pharmaceutical composition for treating Spirochete phylum borne illnesses comprising at least one of tinidazole dissolving bacterial biofilm in combination with minocycline killing a bacterial infection.

9. The pharmaceutical composition of claim 8 further comprising other nitroimidazole class drugs.

10. The pharmaceutical composition of claim 8 further comprising doxycycline.

11. The pharmaceutical composition of claim 8 further comprising coadministering Vitamin D3 or fluoroquinolone.

12. The pharmaceutical composition of claim 8 further comprising pencillin class drugs for patient younger than eight years old preventing teeth staining.

13. The pharmaceutical composition of claim 8 wherein the composition formulating oral administration.

14. The pharmaceutical composition of claim 8 wherein the formulation having a liquid, lotion, cream, spray, gel, ointment, or powder composition.

15. The pharmaceutical composition of claim 8 wherein the formulation administering to an animal patient.

16. The pharmaceutical composition of claim 15 wherein the animal patient is mammalian.

17. The pharmaceutical composition of claim 8 wherein the formulation having a topical lotion, cream, soap, or wipe composition.

18. The pharmaceutical composition of claim 8 wherein the formulation delivery method including oral entailing encapsulation of polypeptide or peptide in microspheres or proteinoids, aerosol, or transdermal.

19. The pharmaceutical composition of claim 18 wherein the oral delivery administering capsules, tablets, gels, pastes, or ointments.

20. The pharmaceutical composition of claim 18 wherein the transdermal administering ointments, cream, lotion or patch.
